(19) United States
(12) Patent Application Publication
(10) Pub. No.: US 2026/0334785 A1
(43) Pub. Date: Oct. 29, 2026

(54) TONOMETER FOR MEASURING PROPERTIES OF EYE AND METHOD THEREOF

(71) Applicant: ICARE FINLAND OY, Vantaa (FI)

(72) Inventors: Andrei Plaian, Noventa Padovana, Pd (IT); Lauri Kangas, Espoo (FI); Matti Raudasoja, Vantaa (FI)

(73) Assignee: ICARE FINLAND OY, Vantaa (FI)

(21) Appl. No.: 19/471,927

(22) PCT Filed: Mar. 27, 2024
(86) PCT No.: PCT/FI2024/050147

§ 371 Date: Oct. 2, 2025

(30) Foreign Application Priority Data
Apr. 25, 2023 (FI) 20235460

(51) Int. Cl. A61B 3/16

(52) U.S. Cl. CPC A61B 3/16

(57) ABSTRACT

A tonometer for measuring properties of an eye. The tonometer has an execution unit including impact means arranged to apply, when the tonometer is in use, an impact on a cornea of the eye; a measuring unit including at least one optical sensor; and a controller coupled to the execution unit and the measuring unit,

wherein the controller is configured, when in use, to operate the execution unit to apply the impact on the cornea of the eye using the impact means; and the at least one optical sensor to perform multiple measurements of a displacement of the cornea caused by the impact during a predetermined time period.

### TECHNICAL FIELD

The present disclosure relates to a tonometer for measuring properties of an eye. The present disclosure also relates to a method for measuring properties of an eye.

### BACKGROUND

Tonometers are medical devices designed to examine an eye of a subject. The eye is examined by measuring various properties thereof. For example, the property is a pressure within the eye, known as intraocular pressure (IOP). The measured eye properties help in diagnosing and managing various eye conditions, including glaucoma, which can lead to vision loss if left untreated. The tonometers can be used for measurement of intraocular pressure (IOP). Typically, tonometers are quick, painless, and non-invasive medical devices.

Notably, there exist various types of tonometers that work on different principles, such as Goldmann applanation tonometer and re-bound tonometers. Goldmann applanation tonometer is typically used as a gold standard on measuring IOP. The measurement however requires relatively long contact with cornea and therefore use of anesthetic substances on the eye are needed. Indeed, measurement with Goldmann applanation tonometer can cause discomfort and potential damage to the corneal surface. Re-bound tonometers have a benefit over Goldmann applanation tonometer as those do not require anesthesia to the eye. In re-bound tonometry a probe or similar is ejected towards a cornea of the eye. Probe speed profile is measured and based on measurements the IOP value is determined.

Therefore, in light of the foregoing discussion, there exists a need to overcome the aforementioned drawbacks associated with existing techniques and devices associated therewith for measuring the eye properties.

### SUMMARY

The present disclosure seeks to provide a tonometer for measuring properties of an eye. The present disclosure also seeks to provide a method for measuring properties of an eye. An aim of the present disclosure is to provide a solution that overcomes at least partially the problems encountered in prior art.

In one aspect, an embodiment of the present disclosure provides a tonometer for measuring properties of an eye, the tonometer comprising:
an execution unit comprising an impact means arranged to apply, when the tonometer is in use, an impact on a cornea of the eye;
a measuring unit comprising at least one optical sensor; and
a controller connected to the execution unit and the measuring unit,
   wherein the controller is configured, when in use, to operate:
   the execution unit to apply the impact on the cornea of the eye using the impact means; and
   the at least one optical sensor to perform multiple measurements of a displacement of the cornea caused by the impact during a predetermined time period.

In another aspect, an embodiment of the present disclosure provides a method for measuring properties of an eye, the method comprising:
creating an impact on a cornea of the eye using impact means, and
performing, using a measuring unit, multiple measurements of a displacement of the cornea caused by the impact during a predetermined time period.

Embodiments of the present disclosure substantially eliminate or at least partially address the aforementioned problems in the prior art, and enable an improved, simple, compact, accurate, reliable and cost-efficient tonometer. This is achieved by causing cornea to oscillate using impact means and measurement of the oscillations using at least one optical sensor. The oscillation parameters such as frequency of the oscillation can be used to determine intraocular pressure of an eye. Indeed, the tonometer employs the at least one optical sensor in order to enable the measurement of the properties of the eye with a better precision.

Additional aspects, advantages, features, and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, similar elements have been indicated by identical numbers.

Embodiments of the present disclosure will now be described, by way of example only, with reference to the following diagrams wherein:

FIG. 1A is a block diagram illustrating a tonometer for measuring properties of an eye, in accordance with an embodiment of the present disclosure;

FIG. 1B is a block diagram illustrating an execution unit of the tonometer, in accordance with an embodiment of the present disclosure;

FIG. 2 is a block diagram illustrating an execution unit of a tonometer, in accordance with another embodiment of the present disclosure;

FIG. 3 is a block diagram illustrating a tonometer, in accordance with the other embodiment of the present disclosure;

FIG. 4 is a block diagram illustrating a tonometer, in accordance with the other embodiment of the present disclosure;

FIG. 5 is a block diagram illustrating at least one optical sensor, in accordance with the other embodiment of the present disclosure;

FIG. 6 is a flowchart depicting steps of a method for measuring properties of an eye, in accordance with an embodiment of the present disclosure;

FIG. 7 is an illustration of oscillations of a cornea caused by impact of the impact means;

FIG. 8 is an illustration of relationship between oscillation frequency and intraocular pressure of an eye;

FIG. 9 is a schematic illustration of a tonometer and its use in accordance with an embodiment of the present disclosure; and

FIG. 10 is illustration of relationship between a damping factor and intraocular pressure of an eye.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a tonometer for measuring properties of an eye, the tonometer comprising:
an execution unit comprising an impact means arranged to apply, when the tonometer is in use, an impact on a cornea of the eye;
a measuring unit comprising at least one optical sensor; and
a controller connected to the execution unit and the measuring unit,
   wherein the controller is configured, when in use, to operate:
   the execution unit to apply the impact on the cornea of the eye using the impact means; and
   the at least one optical sensor to perform multiple measurements of a displacement of the cornea caused by the impact during a predetermined time period.

In another aspect, an embodiment of the present disclosure provides a method for measuring properties of an eye, the method comprising:
creating an impact on a cornea of the eye using impact means, and
performing, using a measuring unit, multiple measurements of a displacement of the cornea caused by the impact during a predetermined time period.

The present disclosure provides the aforementioned tonometer and the aforementioned method that is simple, cost-efficient, robust, accurate, reliable, and user-friendly. Indeed, the impact means are brought in a contact with the cornea of the eye and for a short moment of time. The contact time is less than time it takes for a person to blink the eye. Additionally, the tonometer employs the at least one optical sensor that provides precise measurement of the properties of the eye and in a cost-efficient manner.

Pursuant to the embodiments of the present disclosure, the term “tonometer” as used herein refers to an instrument that is used for measuring various properties of an eye. In this regard, the properties refer to physiological parameters associated with the eye. Optionally, the parameter of the eye measured with the tonometer is intra-ocular pressure.

The term “execution unit” as used herein refers to an arrangement that is used for applying a force (impact) on the cornea of the eye. The term “impact means” as used herein refers to a means that is used for impacting a surface. The impact means is considered as part of the execution unit. In an example, the impact means is used for impacting a corneal surface of the eye. It will be appreciated that the impact means is arranged to create the impact only on the cornea of the eye and without impacting the whole eye together or a volume of tissues surrounding the eye. Typical duration of an impact is in range of 1 to 5 msec depending on a speed and weight of the impact means as well as on properties of the eye (i.e., intraocular pressure value and elasticity of the eye surface, for example). The duration can be between 0.1, 0.5, 1.0, 1.5, 2.0, 2.5, 3.0, 3.5, 4.0, 5.0 up to 0.5, 1.0, 1.5, 2.0, 2.5, 3.0, 3.5, 4.0, 5.0, 6.0 msec. The impact means are applied by launching the impact means towards the eye surface. The impact thus refers to a quick touch/hit/applying of momentary force (an impulse) to the cornea of the eye. The impact is applied on the cornea of the eye in order to make it oscillate. The oscillation of eye due to applied, short duration, impact is referred as free oscillations of the cornea. Beneficially, the duration of impact in range of 1 to 5 msec. In one embodiment the tonometer is implemented as a rebound tonometer, and measurement of oscillations resulting from the impact of a probe of the tonometer, provides an accurate measurement of the internal pressure value, namely, the IOP, of the eye for rebound tonometers. This way one is able to measure IOP using two means, proble velocity profile and also using oscillations resulting from the impact.

The term “measuring unit” as used herein refers to a component that is used for measuring the free oscillations of the cornea of the eye. The measuring unit comprises at least one optical sensor. The at least one optical sensor is used to measure displacement values of the cornea caused by the impact during a predetermined time period. The impact causes the cornea to oscillate. The displacement refers to displacement of the corneal surface from its “normal” level. In practical terms the optical sensor is used to perform multiple (plurality) measurements of the corneal displacements (for example in nanometers) during the predetermined time period. This way a set of displacement (displacement from 0-level) values are collected as function of time for at least the duration of the predetermined time period. The predetermined time period can be time from start of the impact to end time of the oscillations (when the oscillations are damped to level which is insignificant normally about 50 msec after the impact). The predetermined time period can also start during or after the impact. Ideally the predetermined time period lasts as long as one has collected sufficient amount of data points to find frequency of the oscillations. Oscillation frequencies of about 100-500 Hz are anticipated i.e. from this respect the predetermined time is should be in range of at least 1-10 oscillations (1/500 to 10/100 sec). Optionally the predetermined time period is at least 0.001, 0.005, 0.01, 0.015, 0.020, 0.025, 0.030, 0.05, 0.1, 0.15, 0.2, 0.3, 0.5, 1.0 or 2.0 seconds. Since the frequencies are in range up to 500 Hz the at least one optical sensor should be configured to perform in range of 500×2-10 measurements per second i.e. 1000-5000 measurements per second to get sufficient sampling from the displacement. Alternative way to measure displacement is, at the time of impact (or just before), to measure “O-value” and then determine binary value (such as value 1 if the cornea surface is closer to retina than 0-level and value 0 if the cornea surface is further way from the 0-level) if the distance value goes “across” 0-value. This way we can detect oscillation towards retina and away from retina from said 0-value. The term “optical sensor” as used herein refers to an instrument that works by providing a beam of light towards the eye. The beam of light is reflected from the eye surface and the reflection is analyzed to find displacement of cornea at given moment of performing a measurement. As discussed, multiple measurements are done in order to collect sufficient data to determine from the measured oscillation frequency. Moreover, collecting multiple measurements of displacement over time using optical sensors offers numerous benefits, including high temporal resolution that allows for the precise characterization of rapid or transient changes in displacement, dynamic behaviours and responses analysis capabilities, displacement pattern recognition, noise reduction and enhancing the quality of the displacement data, statistical analysis providing quantitative measures of variability, stability, or statistical significance leading to deeper insights, adaptive control to optimize performance, maintain stability, or respond to changing conditions in real time, and long-term monitoring of displacement trends and patterns.

Typically for rebound tonometers, the impact time of the probe with the eye is very short allowing the following corneal oscillation to occur freely without external disruption of a contacting element (impact means such as probe). In case of contact oscillation measurements, the contact element likely directs its own weight and external forces to the cornea that can interfere with the oscillation properties of the eye causing inaccuracy to the measurement results. In addition, their longer contact time with the cornea and longer measurement time potentially causes discomfort to the patient. Thus, contacting the eye only 0.1 to 5 msec using the rebound tonometer and measuring the corneal oscillation in a non-contact manner using an optical sensor, makes the measurement more accurate, shortens the measurement time, and makes the measurement more comfortable for the patient. Measuring the corneal oscillation instead of a simple rebound also improves accuracy compared to present rebound tonometers because more data about the properties of the eye is received at one measure.

Optionally, the at least one optical sensor is selected from at least one of a chromatic confocal sensor, laser doppler vibrometer, a laser displacement sensor. The term “laser doppler vibrometer” as used herein refers to a type of sensor that is used to measure a distance between the tonometer and the cornea of the eye. In this regard, the laser beam from the laser doppler vibrometer is directed at the cornea, and a vibration amplitude and frequency are extracted from a doppler shift of the reflected laser beam frequency due to a motion of the cornea. Typically, the output of the laser doppler vibrometer is generally a continuous analog voltage that is directly proportional to the target velocity component along the direction of the laser beam. The target velocity component can be used to derive, as a measurement value the displacement of the cornea.

The term “laser displacement sensor” as used herein refers to a device that uses a laser beam to measure the distance between two objects. The laser displacement sensor may be designed using a Time-of-Flight (TOF) measurement technique. Optionally, the laser displacement sensor is used to send a laser pulse toward the cornea and measure the time it takes for the pulse to bounce back thereto. The time-of-flight of the laser pulse is then used to determine the distance between the laser displacement sensor and the cornea i.e. to measure displacement

The term “chromatic confocal sensor” refers to device which provides a beam of light with a wide band of wavelengths, usually white light, onto a target surface (such as the eye) using a high dispersion objective lens. Reflected light is analyzed with spectra meter to find which wavelength of the wide band of wavelengths has a peak in the spectra. The peak wavelength corresponds to distance between the sensor and the reflecting surface (eye surface). Again, as in previous example, by performing multiple (plurality) measurements in a row we can collect sufficient data set of displacements of the cornea as function of time. This data can be used to find oscillation frequency of the eye. As an example, number of measurements per second can be, as discussed, 1000 to 5000 per second for example or more depending on the accuracy requirement. For example, 10 k-20 k measurements per second can be used.

The measuring unit includes one or more optical sensors. Optionally, the measuring unit includes multiple optical sensors in order to improve the accuracy of the IOP measurement. For example, different areas of the cornea can have different biomechanical properties. In such a case, the multiple optical sensors could measure the cornea from multiple angles or locations and provide a more information to be used measurement of properties of an eye.

Optionally, the at least one optical sensor comprises at least one light emitting diode (LED) and at least one photosensor, wherein the at least one photosensor is selected from at least one of: a phototransistor or a photodiode. The term “light emitting diode” as used herein refers to a semiconductor device that emits light when an electric current flows therethrough. Moreover, each of the at least one optical sensor comprises the LED as a light source thereof. The photons emitted by the LED are directed towards a target object such as the cornea of the eye. When the photons hit the cornea, some of the photons are reflected back toward the at least one optical sensor. Optionally, the amount of light reflected back to the at least one optical sensor depends on the reflectivity of the cornea of the eye. Benefit of using LED's instead of laser is that LEDs are not mono chromatic. Monochromatic light from laser might have adverse effect on retina. LED's provide relatively (to laser) broad spectrum of light.

The term “phototransistor” as used herein refers to a type of semiconductor device that is used to detect light. Moreover, each of the at least one optical sensor comprises the phototransistor that works in conjunction with the LED. The term “photosensor” as used herein refers to a device that detects light and converts it into an electrical signal. Moreover, the photosensor works in conjunction with the LED present in each of the at least one optical sensor. Optionally, the photosensor is a photodiode that is a semiconductor device sensitive to light. Both phototransistors and photodiodes are fast components and thus suitable for measuring fast transients and for example pulsed light sources accurately. According to an embodiment, the measured displacement of the cornea does not need to be an absolute value (such as nanometers) but it is sufficient to find time evolution of the displacement. In this regard, using a pair of LED and photosensor can provide photocurrent as function of time as output. This photocurrent does not provide direct information of actual amount of displacement (in meters). However, the photocurrent as function of the time, can be considered to represent relative displacement (between different moments of times) and can be used in further data processing to find the frequency of oscillations. Moreover, LED and photodiode sets are non-invasive and can be comfortably placed near the eye without causing discomfort or interfering with normal vision. Additional benefit of using LED and photosensor/photo transistor is inexpensive nature of said components.

It will be appreciated that the at least one optical sensor is placed at an appropriate position, for identifying the oscillations of the cornea accurately. Preferably, the optical sensor is arranged close to execution unit, and preferably, arranged to measure from same direction than the impact means are applied to the cornea of the eye. Optionally, the beam of light is preferably oriented normal to the cornea. Advantageously, the at least one optical sensor possesses the ability to tolerate a large inclination of an axis of the beam with respect to the normal direction of the measured surface (even tens of degrees). It will be appreciated that the at least one optical sensor is used for resolutions of measurement of the displacement of tens of nanometres with capability to measure with frequency of tens of kilohertz (kHz). Beneficially, the at least one optical sensor is customized to identify the frequency of oscillations of the cornea in order to make it cost-efficient. Optionally, the at least one optical sensor uses infrared light (IR) as a measuring beam. Beneficially, the IR light is not seen by the eye of the subject. In such a case, the temptation of the subject to blink the eye is reduced.

The term “controller” refers to a computational device that is operable for controlling the overall operation of the tonometer. The controller, in operation, performs tasks such as, but not limited to, using the execution unit, using the measuring unit and responding to and processing information. In an example, the controller may be an embedded microcontroller, a microprocessor, and the like. In this regard, the controller is coupled with the measuring unit and the execution unit. The controller may be implemented as an internal component of the tonometer, an external component of the tonometer, or a combination thereof.

The controller is configured to operate the execution unit by energizing thereto. In this regard, the execution unit is energized in order to apply the impact meaning a force on the cornea of the eye. Notably, the eye is considered to be a thin-walled elastic vessel filled with a pressured fluid. As a consequence, the eye has a mechanical vibrational behaviour and a resonance frequency. Moreover, when the cornea experiences the impact, it will deform and then rapidly return to its original shape, creating oscillations in its surface. The oscillation is the evolution in time of the displacement of the surface of the cornea. The term displacement refers to amplitude of oscillations.

Herein, the multiple measurements refer to one or more measurements of the displacement of the corneal over a predetermined period of time. In this regard, the multiple measurements provide more accurate and reliable data, as they can help account for any variability or inconsistency in the measurements. Optionally, the multiple measurements enable tracking of a progression of the displacement of the cornea over time and get a better understanding of the impact's effects on the cornea. Herein, the displacement refers to a change in a position of the cornea caused by the oscillations thereof. The frequency of the multiple measurements is high enough to identify the oscillations of the cornea.

The controller is configured to operate or energize the measuring unit. In this regard, the controller operates the measuring unit having the at least one optical sensor to measure the displacement of the cornea. As an example, the photosensor comprises a LED as light emitter. The LED of the optical sensor sends (emits) A beam of light onto the cornea, and the corresponding light detector (such as at least one photosensor) measures the amount of light reflected back from the cornea to measure displacement of the cornea. In this example as the impact means hit the cornea the cornea is slightly deformed. For this reason, the distance between the at least one LED and the at least one photosensor changes, causing a change in the amount of light reflected back to the at least one photosensor. The change in the amount of reflected light can be used as the measured displacement (displacement value such as X meters (example −150 to 150 micrometers) from “O-level” of corneal surface or as change in current in photosensor). Herein, the measuring unit is used for performing multiple measurements of the displacement of the cornea that is caused by the impact during the predetermined time period.

Optionally, the tonometer further comprises a calculating unit coupled to the controller and operable to
use the measured displacements to calculate a frequency of free oscillations of the cornea caused by the impact; and
use the frequency of the free oscillations of the cornea to calculate an internal pressure value of the eye (which the internal pressure value can be assumed to be a function of the frequency of the free oscillations of the cornea caused by the impact). Indeed, the measured displacements (such as amplitude from “zero” level) as a function of time provide way to find oscillation frequency. Frequency of free oscillations can be calculated from the measured displacements by finding periodicity of the measurements i.e. for example time difference between two peaks or time difference between multiple peak pairs, calculating average of those. Frequency is thus inverse of said found average time difference (1/s=Hz). The internal pressure of the eye can be found from the calculated frequency of free oscillations (non-disturbed oscillations) for example by using look up table having correlation table of frequency vs intraocular pressure (IOP). I.e the frequency of the free oscillations of the cornea is used to calculate an internal pressure value of the eye. Alternative way to calculate is to use mathematical model of eye oscillations and use the frequency as input value for the mathematical model.

Further, optionally, the tonometer further comprises a calculating unit coupled to the controller and operable to
use measured displacements to calculate a damping factor of the free oscillations of the cornea caused by the impact; and
use the damping factor to calculate an internal pressure value of the eye.

Additionally, the tonometer is configured to adjust internal pressure values as follows:
the calculated internal pressure value of the eye using the frequency of the free oscillations is adjusted, using the calculated internal pressure value of the eye using the damping factor, or
the calculated internal pressure value of the eye using the damping factor is adjusted, using the calculated internal pressure value of the eye using the frequency of the free oscillations.

The term “calculating unit” as used herein refers to an electronic hardware or a software algorithm that is used for performing mathematical operations to convert raw data into a meaningful measurement. In this regard, the calculating unit is associated with the controller in order to allow for greater flexibility and customization in the tonometer based on a design thereof. Optionally, the calculating unit is integrated into the controller i.e., it is part of the controller. Optionally, the calculation unit is implemented as cloud service. Moreover, the calculating unit is operable use the measured displacement (values or indicators of the displacement) to calculate a frequency of free oscillations of the cornea. The free oscillations refer to a resonance frequency or natural oscillation frequency of the cornea, that is induced by the impact. In practice, the cornea performs the free oscillations with a frequency that equals (or is close) to its resonance frequency with a decreasing amplitude until it stops. One way to calculate the frequency is using inverse Fourier transformation. The frequency of the free oscillations is correlated to the internal pressure of the eye and thus by determining/measuring the frequency the internal pressure (intraocular pressure IOP) value of the eye can be measured. Another way to calculate the frequency is by comparing “O-level” crossings obtained with the optical sensor. This simplifies process as we can calculate easily time of changes from 1 (indicating corneal surface to be closer to retina than it normally is) and 0 (indicting corneal surface to be further away from retina than it normally is) and from 1 to 0.

As discussed above, the cornea oscillates with the frequency of free oscillations. The amplitude of the oscillations decreases over time (of few msecs up to 100 msec.) This decrease of the amplitude can be described using a damping factor of the oscillations. Furthermore, the measured multiple displacement values (as function of time) can be used to calculate internal pressure value of the eye. This way by performing multiple (plurality) measurements of a displacement of the cornea caused by the impact, IOP (intra ocular pressure) can be measured (indirectly) via the damping factor. In general eye can be considered to be a harmonic oscillator. Basic equation of the harmonic oscillator is:

$$\frac{{d}^{2} x}{{dt}^{2}} + 2 {ζω}_{0} \frac{dx}{dt} + {ω}_{0}^{2} x = 0$$

wherein x is the measured displacement value (from normal level) i.e., amplitude, t is time, ω is frequency and ζ is the damping factor. This can be solved to find displacement as function of time x(t):

$$x ( t ) = \frac{1}{ω} {e}^{- {ζω}_{0} t} sin ω t$$

Indeed, by performing multiple displacement value measurements over a period of time we can obtain data points x(t). These can be used to calculate frequency and the damping factors.

Based on experiments, the damping factor can be used, in addition to the frequency, to define the internal pressure value of the eye. This can be done for example by curve fitting, using lookup table or predetermined equation. Using the damping factor is beneficial since error margin related to damping factor determination is small. Alternative way to calculate damping factor is to find from the performed multiple measurements of displacement peak maximums and minimums of each oscillation. The peak maximums and minimums can be used to find damping by comparing for example two different peak maximums.

According to an alternative embodiment, the internal pressure value of the eye is calculated using the frequency of the free oscillations and that value is adjusted based on the internal pressure value which is calculated using the damping factor. According to a further alternative embodiment, the internal pressure value of the eye is calculated using the damping factor and that value is adjusted based on the internal pressure value which is calculated using the frequency of the free oscillations of the cornea. This way we can improve accuracy of the measurements.

The term “internal pressure” (namely an intraocular pressure (IOP)) as used herein refers to a fluid pressure of the eye. Optionally, the calculating unit may apply different mathematical models, such as regression analysis or curve fitting, to the raw data to obtain the most accurate and reliable measurements of the internal pressure value of the eye. Optionally, the calculating unit may perform other functions, such as error correction, data storage, and data analysis. Notably, the internal pressure value of the eye is determined to maintain overall eye health and function. Optionally, the eye presents a mass-spring system, where the IOP describes the spring constant. Optionally, when the cornea is displaced from its natural position by an external force, it vibrates as a damped harmonic oscillator with a frequency relative to the IOP. The IOP modulates the stiffness of the eye as well as the cornea so that the oscillation frequency increases with increasing IOP. It will be appreciated that the measurement of the internal pressure value enables a diagnosis and treatment of ocular hypertension before the development of eye-related conditions. Moreover, the dimensions are determined for diagnosing eye growth regulation and the touch sensitivity is determined for treatment of eye-related conditions such as conjunctivitis, corneal infections, glaucoma, dry-eye and so forth.

The frequency and damping factor of the free oscillations of the cornea can provide valuable information for evaluating the IOP based on the biomechanical properties of the cornea, including corneal stiffness and elasticity, that can influence how the cornea responds to changes in IOP. Notably, corneal thickness can vary among individuals and can affect the accuracy of IOP measurements. By considering corneal biomechanical properties such as frequency and damping factor, IOP estimates that are less dependent on corneal thickness, can be obtained using simple tonometers, potentially improving accuracy, especially in patients with abnormal corneal thickness. Moreover, IOP calculation based on frequency and damping factor of the free oscillations of the cornea provide a more holistic understanding of the factors contributing to ocular pathology and develop tailored treatment strategies accordingly. For example, changes in corneal biomechanics, including alterations in the frequency and damping factor of corneal oscillations, may be monitored over time to help in the early detection and management of glaucoma or corneal ectasia by providing additional indicators of ocular health, disease progression or treatment effectiveness beyond traditional IOP measurements, ultimately improving clinical decision-making and patient care.

Optionally, the impact means of the execution unit is a sold probe and the execution unit further comprises a launching means, wherein the launching means is operable to launch the solid probe towards the eye to create the impact.

The term “launching means” as used herein refers to a mechanical element that is used to release the at least one solid probe from the execution unit towards (or in the direction of) the eye. In this regard, the launching means enables an efficient and accurate movement of the solid probe, when in use. As an example, the solid probe can be an elongated probe having a magnetic elongated body and a bio compatible tip part. In this example, the launching means partially surrounds the magnetic elongated body of the solid probe. Herein, the launching means is arranged as a set of loops (of an electric coil) through which the at least one solid probe can move. The launching means moves the at least one solid probe when the electric current is fed through the set of loops means. The electric current creates a magnetic field on the loops which moves the probe (since it has magnetic elongated body arranged at least partially inside of the loops). The launching means ejects the solid probe towards the eye with a velocity which is a function of the electric current fed through the loops of the launching means and the magnetization of the body of the solid probe. It will be appreciated that the launching means and the at least one solid probe works in conjunction with each other to create an accurate impact on the cornea of the eye meaning that on the right point on the cornea.

Optionally, the impact means of the execution unit is at least one drop of liquid and the execution unit further comprises a launching means, wherein the launching means is operable to launch the at least one drop of liquid towards the eye to create the impact.

Herein, the launching means refers to another mechanical element that is used to release or launch one or more drops of the liquid towards the eye to create the impact on the cornea of the eye. Optionally, the at least one drop of liquid is selected from at least one of: water, a salted water or a physiological solution. Optionally, the at least one drop of liquid is water. Optionally, the drop of water could be a non-irritating liquid that can be launched towards the eye without causing harm or discomfort thereto. Optionally, the at least one drop of liquid is the salted water. Typically, the salted water is a sterile solution of water and salt that is used for various purposes such as an eye irrigation. The eye irrigation is a process of flushing out the eye with a stream of fluid in order to remove foreign objects, irritants, or chemicals that may have entered the eye. Optionally, the at least one drop of liquid is a physiological solution. In this regard, when in operation, the controller is configured to operate the execution unit in such a manner that the launching means associated therewith launches the at least one drop of liquid towards the eye in order to create oscillations in the cornea. The frequency of the oscillation is indicative of the value of the IOP as discussed. Notably, a higher IOP is indicative of an increased stiffness of the eyeball, and therefore an increased resonance frequency of the cornea. In an alternative or additional embodiment, the liquid can be a medicine and the tonometer is arranged to administer the medicine. Measurement can be used to detect if the medicine is administered or not (by detecting with the optical sensor if there is movement caused by administered medicine in the cornea or not).

Optionally, the at least one drop of liquid is stored in a hollow container. Optionally, the hollow container is arranged inside the at least one solid probe which is associated with the launching means. It will be appreciated that the launching means works in conjunction with the at least one solid probe in order to deliver an optimum quantity of the at least one drop of liquid at the cornea. Beneficially, when the at least one drop of liquid is delivered in the optimum quantity it creates an accurate impact on the cornea of the eye.

Beneficially, applying at least one drop of liquid to the eye is a non-invasive method for inducing corneal oscillations without causing discomfort or harm to the patient and/or minimal risk of adverse effects. Moreover, the application of at least one drop of liquid to induce corneal oscillations can be standardized and reproducible across different patients to ensure consistency in experimental protocols and facilitates comparisons between studies. Furthermore, by controlling the volume and composition of the drop of liquids, the amplitude and frequency of corneal oscillations induced in the eye may be regulated. This allows for precise manipulation of the experimental conditions, such as external stimuli or fluctuations in ocular conditions, to study their effects on IOP dynamics, such as glaucoma, and help identify biomarkers or patterns indicative of disease progression or treatment response.

Optionally, the tonometer further comprises
at least one sensor element operable to measure a distance between the eye and the tonometer, and
a feedback element, coupled to the at least one sensor element, operable to provide a feedback based on the measured distance;
   wherein the feedback is selected from at least one of: a visual feedback, an acoustic feedback or a feedback signal.

Herein, the sensor element refers to a device that detects and responds to physical changes in its environment. Herein, the tonometer comprises one or more sensors that are used to convert physical phenomena, such as light, motion, and so forth into electrical or digital signals that can be measured or processed. Optionally, the at least one sensor is an internal component of the tonometer or an external component of the tonometer. Optionally, the at least one sensor element can be used alone or in combination with other sensors to provide more complex and detailed measurements of the environment or system being monitored. The term “feedback element” as used herein refers to an electronic component that provides information about an output of a sensor and allows for adjustments to be made to the sensor's input or output in response to that information. Optionally, the feedback element could be a physical component, such as a potentiometer, or a digital component, such as a microcontroller. It will be appreciated that the feedback element is used to improve the accuracy and reliability of the at least one sensor element by detecting errors or discrepancies between the desired and the measured distance values, and making adjustments to correct them.

In this regard, when in operation, the at least one sensor element is operable to measure the distance between the cornea and the tonometer and provide the measured distance as the feedback to the feedback element coupled therewith. The feedback element compares the measured distance of the at least one sensor element to a reference value (such as the desired or the optimum distance), and the controller adjusts the input to the sensor based on the difference between the two values. It will be appreciated that the at least one sensor works in conjunction with feedback element to correctly position the tonometer relative to the eye, when the tonometer is a hand-held unit. Moreover, the feedback is provided as the visual feedback. For example, the measured distance is displayed on a screen of a device. Optionally, the visual feedback is represented in the form of a textual information or a graphical information. Furthermore, the feedback is provided as the acoustic feedback. For example, the measured feedback is provided to an operator of the tonometer as an audio feedback via a speaker associated with a device. Advantageously, the visual feedback or the acoustic feedback is used in order to assist the correct positioning of the tonometer with respect to the eye. Furthermore, the feedback can be provided as a feedback signal. The feedback signal is an electrical signal (such as command, or voltage value or a bit stream) which can be provided to other electrical components to control those or provide a control command to those.

Optionally, the tonometer further comprises:
at least an actuator, operable to automatically change the relative position of the tonometer with respect to the cornea of the eye based on the feedback signal.

The term “actuator” as used herein refers to a component that is responsible for automatically changing the relative position of the tonometer with respect to the eye, based on the feedback signal received from the feedback element. Optionally, this could involve moving the tonometer closer or further away from the eye, or adjusting its angle or orientation. Examples of actuators include motors, solenoids, and piezoelectric devices. In this regard, the tonometer is positioned with respect to the eye by means of the at least one actuator, in which a position feedback signal is obtained by measuring the distance between the tonometer and the eye by means of the at least one sensor element. For example, if the measured distance is more than the desired distance then the feedback element is operable to provide the feedback signal to the actuator and then the actuator is operable to move the tonometer at an optimum position with respect to the eye.

Optionally, the at least one sensor element is selected from a laser displacement sensor. In this regard, the tonometer is associated with one or more sensor elements that are useful to give a position feedback. The position feedback is provided at least in an initial phase of positioning of the tonometer relative to the cornea, before the cornea enters inside the measuring field of the given optical sensor. Optionally, when the cornea is inside the measuring field of the given optical sensor it is possible to either use the given optical sensor to get the position feedback or to continue using the laser displacement sensor. Beneficially, the laser displacement sensor is a low-cost sensor.

The disclosed tonometer enables measuring a displacement of the cornea for the whole or part of the free oscillation period in order to determine accurate intraocular pressure of the eye by employing contact tonometry, i.e., using a tonometer implemented as a rebound tonometer. In this regard, the tonometer uses an execution unit comprising impact means arranged to apply, when the tonometer is in use, an impact on a cornea of the eye, wherein the impact is in range of 0.1 to 5 msec and a measuring unit comprising at least one optical sensor. When the rebound tonometer is in use, the probe of the rebound tonometer hits the cornea of the eye causing the displacement of the cornea of the eye and then free oscillations are measured. However, the probe hits the cornea of the eye only for a short impact time and the contact period is not long enough to measure corneal oscillation in order to obtain an accurate internal pressure value of the eye. Moreover, the measurement with the optical sensor allows to measure the displacement of the of the cornea of the eye for an extended period of time without being in contact with the eye, this allows more accurate measurements and in turn also more accurate internal pressure value of the eye. Furthermore, the use of optical sensors solves a technical problem of how to measure a displacement of the cornea for the whole free oscillation period in order to determine accurate intraocular pressure of the eye with a rebound tonometer (i.e., a contact tonometer) without requiring a non-contact tonometer.

The present disclosure also relates to the method as described above. Various embodiments and variants disclosed above, with respect to the aforementioned first aspect, apply mutatis mutandis to the method.

According to embodiments property of an eye is measured by creating an impact on a cornea of the eye using impact means. The impact will cause the cornea to oscillate. According to the method, multiple measurements of a displacement (related to the corneal oscillation) are performed during a predetermined time period. The predetermined time period is for example the duration of the oscillations. Multiple measurements are done in order to collect displacement (value/indicator) as function of time. Impact can be created using impact means of the execution unit. The displacement measurements can be done using at least one optical sensor.

Optionally, the method further comprises
calculating a frequency of free oscillations of the cornea caused by the impact using the multiple measurements of the displacement of the cornea; and
calculating an internal pressure value of the eye from the frequency of the free oscillations of the cornea caused by the created impact. The internal pressure value of the eye is function of measured frequency. The value can be determined using (a device specific) function or look up table.

Optionally, performing multiple measurements of the displacement is done using at least one optical sensor is, the optical sensor is selected from at least one of: a confocal chromatic sensor, a laser doppler vibrometer or a laser displacement sensor.

Optionally, the at least one optical sensor comprising at least one light emitting diode (LED) and at least one photosensor, wherein the at least one photosensor is selected from at least one of: a phototransistor or a photodiode.

Optionally, the method further comprises
measuring a distance between the cornea and the tonometer, and
using the measured distance as a feedback signal for moving the execution unit at a desired position relative to the cornea.

Optionally, the impact on the cornea is created by releasing the at least one probe towards the eye, the at least one solid probe being comprised in the execution unit.

Optionally the method comprises calculating a damping factor of the free oscillations of the cornea caused by the impact using the multiple measurements of the displacement of the cornea; and calculating an internal pressure value of the eye using the calculated damping factor.

According to one embodiment a tonometer for measuring properties of an eye is provided. The tonometer comprising:
an execution unit comprising impact means arranged to apply, when the tonometer is in use, an impact on a of the eye, wherein duration of the impact is in range of 0.1 to 5 msec;
a measuring unit comprising at least one optical sensor;
a calculating unit; and
a controller coupled to the execution unit, the measuring unit and the calculating unit,
   wherein the controller is configured, when in use, to operate:
   the execution unit to apply the impact on the cornea of the eye using the impact means;
   the at least one optical sensor to perform multiple measurements of a displacement of the cornea caused by the impact during a predetermined time period; and
   the calculating unit to use the measured displacements to calculate a frequency of free oscillations of the cornea caused by the impact and to use the frequency of the free oscillations of the cornea to calculate an internal pressure value of the eye or
   the calculating unit to use measured displacements to calculate a damping factor of the free oscillations of the cornea caused by the impact and use the damping factor to calculate an internal pressure value of the eye, wherein the impact means of the execution unit is a solid probe and the execution unit further comprises:
a launching means,
   wherein the launching means is operable to launch the solid probe towards the eye to create the impact. Alternatively, combination of frequency of free oscillations and the damping factor can be used to calculate the internal pressure value of the eye.

### DETAILED DESCRIPTION OF THE DRAWINGS

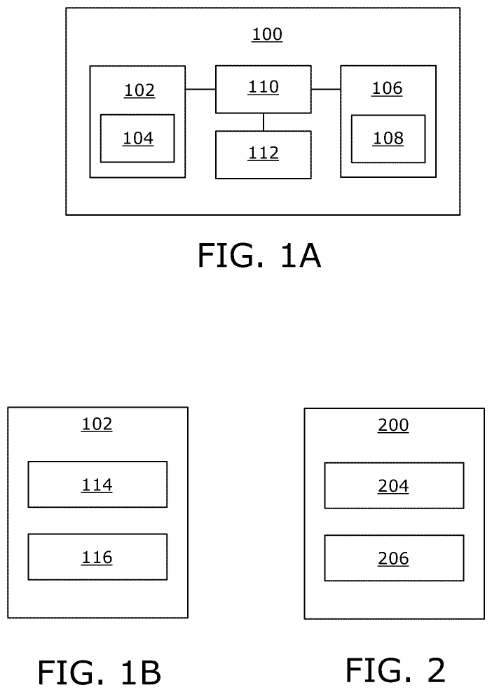

Referring to FIG. 1A, there is shown a block diagram illustrating a tonometer 100 for measuring properties of an eye, in accordance with an embodiment of the present disclosure. As shown, the tonometer 100 comprises an execution unit 102 comprising an impact means 104 arranged to apply an impact on a cornea of the eye. Moreover, the tonometer 100 comprises a measuring unit 106 comprising at least one optical sensor 108 and a controller 110 coupled to the execution unit 102 and the measuring unit 106. It will be appreciated that the controller 110 is configured to operate the execution unit 102 to apply the impact on the cornea of the eye using the impact means 104 and the at least one optical sensor 108 to perform multiple measurements of a displacement of the cornea caused by the impact during a predetermined time period. Furthermore, the tonometer 100 comprises a calculating unit 112 coupled to the controller 110 and operable to calculate a frequency of free oscillations of the cornea caused by the impact using the multiple measurements of the displacement of the cornea and calculate an internal pressure value of the eye as a function of the frequency of the free oscillations of the cornea caused by the impact.

Referring to FIG. 1B, there is shown a block diagram illustrating the execution unit 102 of the tonometer 100, in accordance with an embodiment of the present disclosure. As shown, the execution unit 102 further comprises a launching means 114, and at least one solid probe 116 and wherein the launching means 114 is operable to launch the at least the one solid probe 116 towards the eye to create the impact.

FIGS. 1A and 1B are merely examples, which should not unduly limit the scope of the claims herein. A person skilled in the art will recognize many variations, alternatives, and modifications of embodiments of the present disclosure.

Referring to FIG. 2, there is shown a block diagram illustrating an execution unit 202 of a tonometer 200, in accordance with another embodiment of the present disclosure. As shown, the execution unit 202 further comprises a launching means 204, and at least one drop of liquid 206, and wherein the launching means 204 is operable to launch the at least the one drop of liquid 206 towards the eye to create the impact.

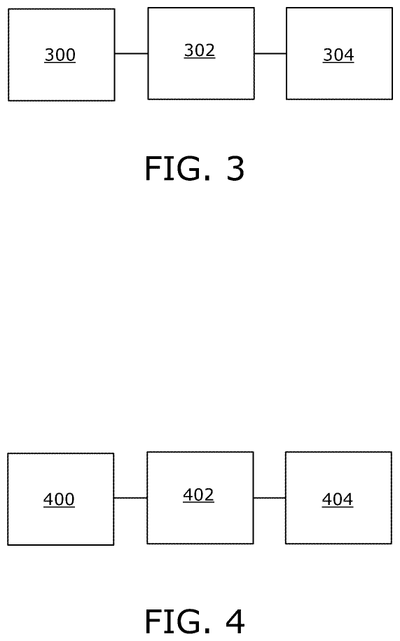

Referring to FIG. 3, there is shown a block diagram illustrating a tonometer 300, in accordance with the other embodiment of the present disclosure. Optionally, the tonometer 300 further comprises at least one sensor element 302 operable to measure a distance between the cornea of the eye and the tonometer 300, and a feedback element 304, coupled to the at least one sensor element 302, operable to provide a feedback based on the measured distance. Optionally, the feedback is selected from at least one of: a visual feedback, an acoustic feedback, a feedback signal.

Referring to FIG. 4, there is shown a block diagram illustrating a tonometer 400, in accordance with the other embodiment of the present disclosure. Optionally, the tonometer 400 further comprises at least one sensor element 402 operable to measure a distance between the eye (not shown) and the tonometer 400, Moreover, optionally, the tonometer 400 further comprises at least an actuator 404, operable to automatically change the relative position of the tonometer 400 with respect to the cornea of the eye, wherein the measured distance between the tonometer 400 and the cornea is used as a feedback signal to automatically control the relative position between the tonometer 400 and the cornea.

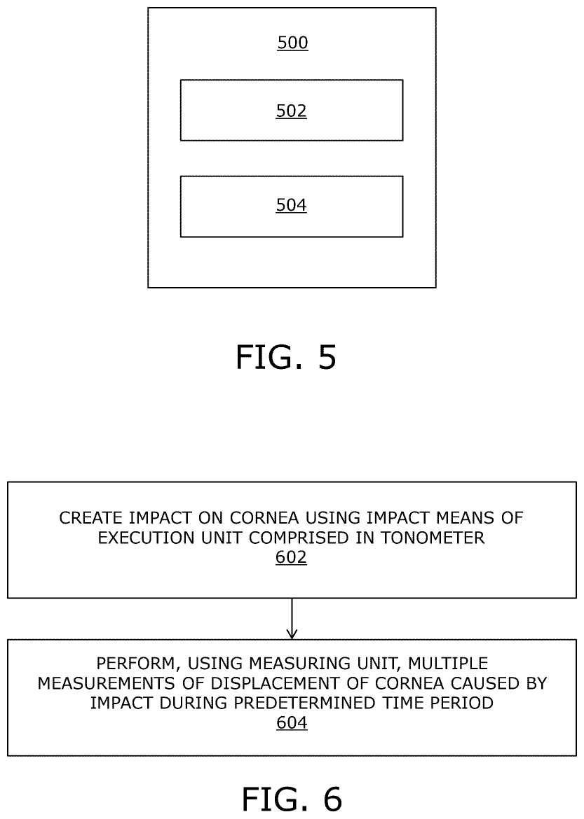

Referring to FIG. 5, shown is a block diagram illustrating at least one optical sensor 500, in accordance with an embodiment of the present disclosure. As shown, the at least one optical sensor 500 comprises at least one LED 502 and the at least one photosensor 504. Optionally, the at least one photosensor 504 is selected from at least one of a phototransistor. a photodiode.

FIGS. 2, 3, 4 and 5 are merely examples, which should not unduly limit the scope of the claims herein. A person skilled in the art will recognize many variations, alternatives, and modifications of embodiments of the present disclosure.

Referring to FIG. 6, shown is a flowchart depicting steps of a method for measuring properties of an eye, in accordance with an embodiment of the present disclosure. At step 602, an impact on a cornea is created using impact means of execution unit comprised in a tonometer. At step 604, multiple measurements of a displacement of the cornea caused by the impact during a predetermined time period is performed using a measuring unit.

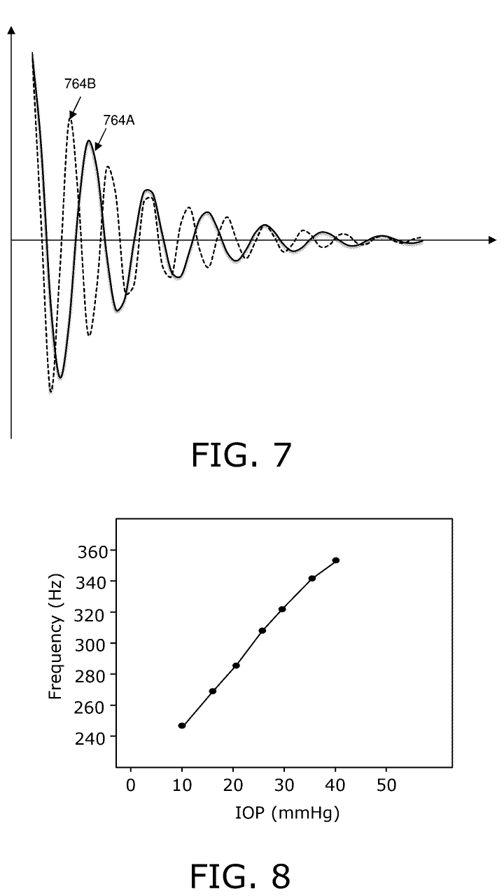

FIG. 7 is an illustration of a measurement of displacement of cornea caused by the impact during a predetermined time period. X-axis is a time in arbitrary units. Y-axis is displacement in arbitrary units. As it can be seen frequency of a first set of measurements 764A (solid line) has lower frequency than a frequency of a second set of measurements 764B (dotted line). Said graph can be obtained by performing multiple measurements of displacement as function of time. For example, if the total time of the predetermined time period is 50 msec then number of measurements during said time could be for example 20, 50, 100, 1000 or 10000. The said measurements can be used to find frequency of the oscillations.

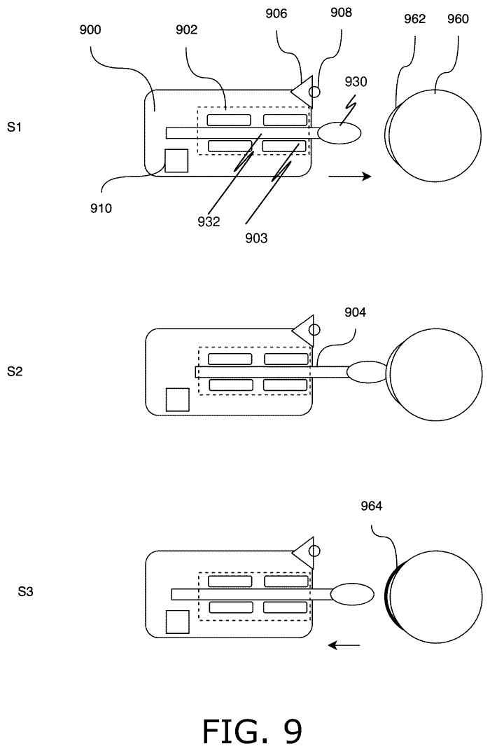

FIG. 8 is an illustration of correlation of the frequency of oscillations (Hz) to intraocular pressure value (mmHg). It can be seen that larger frequencies indicate larger pressure values and vice versa. FIG. 9 is illustration of steps of using a tonometer 900 according to embodiments of present disclosure. The tonometer 900 comprises an execution unit 902. The execution unit comprises impact means 904. In the figure the impact means is a solid probe having a tip part 930 connected to elongated magnetic body 932. The elongated magnetic body is partly surrounded with electrical loops 903 of the execution unit 902.

A measuring unit 906 is attached to body of the tonometer 900 in such a way that it is directed towards eye 960 when the tonometer is in use. The measuring unit comprises at least one optical sensor 908. A controller 910 is coupled to the execution unit 902 and the measuring unit 906. In step S1 the controller operates the execution unit 902. In this example the electrical loops 903 are energized. Current flowing thru the electrical loops 903 creates an electric field which in in turn creates magnetic force to the elongated magnetic body 932 of the impact means 904. This force launces the impact means towards (as indicated with arrow) cornea 962 of the eye 960. In step S2 the impact means 904 is illustrated to apply the impact on the cornea 962. i.e. the tip part 930 collides with the cornea 962 of the eye 960.

Due to elasticity of the eye 960 the impact means 904 bounces back after the applied impact as illustrated with an arrow in step S3. According to alternative embodiment the execution unit 902 can be configured to actively pull back the impact means 904 after the impact. This can be done by reversing direction of electrical current in the electrical loops 903 in respect to launch direction. After the impact the cornea 962 oscillates. Oscillations 964 are indicated with thickened line in the figure. The optical sensor 908 of the measuring unit 906 performs multiple measurements of a displacement of the oscillations caused by the impact during a predetermined time period. The measurements are stored in a memory of the controller 910 for further data processing. The controller can comprise a calculation unit (or there can be a separate calculation unit). The calculation unit is used to use the stored measurement values and use those to calculate frequency of free oscillations (for example by Fourier analysis). Furthermore, the frequency is used to calculate (using look up table or equation) the internal pressure value of the eye.

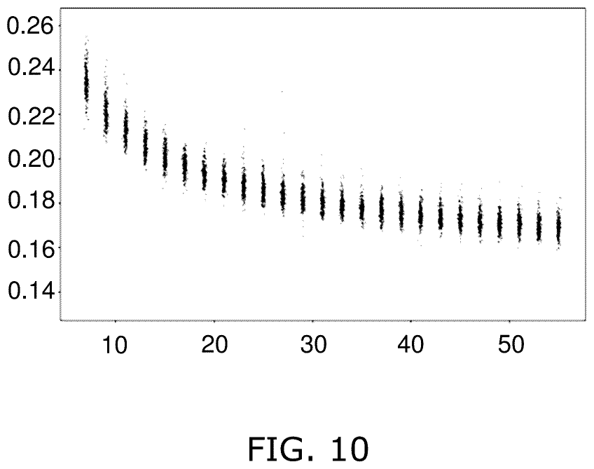

FIG. 10 is an illustration of relationship between a damping factor of the oscillations (decrease of the amplitude over time) from a set of performed experiments. x-axis of the graph is intraocular pressure of the eye (in mmHg) and y-axis is the damping factor. It can be seen that particularly with lower pressures (in range of below 20 mmHg) the damping factor provides a good indication of the pressure with good confidence level. Indeed, damping factor might provide better adjustment (calibration/correction) to IOP measured with the frequency in said low range than in higher range. According to one embodiment damping factor is used if IOP is below 15, 20, 25, 30 or 40 mmHg as the IOP value or to adjust the IOP value measured using the frequency of the free oscillations. Beneficially, the disclosed tonometer may be calibrated regularly to ensure accuracy, help maintain the reliability of the measurements and ensure that the tonometer is functioning correctly.

The aforementioned steps are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A tonometer for measuring properties of an eye, the tonometer comprising:
an execution unit comprising impact means arranged to apply, when the tonometer is in use, an impact on a cornea of the eye;
a measuring unit comprising at least one optical sensor; and
a controller coupled to the execution unit and the measuring unit,
wherein the controller is configured, when in use, to operate:
the execution unit to apply the impact on the cornea of the eye using the impact means; and
the at least one optical sensor to perform multiple measurements of a displacement of the cornea caused by the impact during a predetermined time period.

2. A tonometer according to claim 1, further comprising a calculating unit coupled to the controller and operable to
use the measured displacements to calculate a frequency of free oscillations of the cornea caused by the impact; and
use the frequency of the free oscillations of the cornea to calculate an internal pressure value of the eye.

3. A tonometer according to claim 1, further comprising a calculating unit coupled to the controller and operable to
use measured displacements to calculate a damping factor of the free oscillations of the cornea caused by the impact; and
use the damping factor to calculate an internal pressure value of the eye.

4. A tonometer according to claim 2, wherein:
the calculated internal pressure value of the eye using the frequency of the free oscillations is adjusted, using the calculated internal pressure value of the eye using the damping factor or
the calculated internal pressure value of the eye using the damping factor is adjusted, using the calculated internal pressure value of the eye using the frequency of the free oscillations.

5. A tonometer according to claim 1, wherein the at least one optical sensor is selected from at least one of: a chromatic confocal sensor, a laser doppler vibrometer or a laser displacement sensor.

6. A tonometer according to claim 1, wherein the at least one optical sensor comprises at least one light emitting diode and at least one photosensor, and wherein the at least one photosensor is selected from at least one of: a phototransistor or a photodiode.

7. A tonometer according to claim 1, wherein the impact means of the execution unit is a solid probe and the execution unit further comprises a launching means wherein the launching means is operable to launch the solid probe towards the eye to create the impact.

8. A tonometer according to claim 1, wherein the impact means of the execution unit is at least one drop liquid, and the execution unit further comprises
a launching means; and
and wherein the launching means is operable to launch the at least one drop of liquid towards the eye to create the impact.

9. A tonometer according to claim 8, wherein the at least one drop of liquid is selected from at least one of: water, a salted water or a physiological solution.

10. A tonometer according to claim 1, further comprising
at least one sensor element operable to measure a distance between the eye and the tonometer; and
a feedback element, coupled to the at least one sensor element, operable to provide a feedback based on the measured distance;
wherein the feedback is selected from at least one of: a visual feedback, an acoustic feedback or a feedback signal.

11. A tonometer according to claim 10 further comprising at least an actuator operable to automatically change the relative position of the tonometer with respect to the cornea of the eye based on the feedback signal.

12. A tonometer according to claim 10, wherein the at least one sensor element is a laser displacement sensor.

13. A tonometer according to claim 1 wherein a duration of an impact is between 0.1 to 5 msec.

14. A method for measuring properties of an eye, the method comprising:
creating an impact on a cornea of the eye using impact means and
performing, multiple measurements of a displacement of the cornea caused by the impact during a predetermined time period.

15. A method according to claim 14, further comprising
calculating a frequency of free oscillations of the cornea caused by the impact using the multiple measurements of the displacement of the cornea; and
calculating an internal pressure value of the eye from the frequency of the free oscillations of the cornea caused by the created impact.

16. A method according to claim 14, further comprising
calculating a damping factor of the free oscillations of the cornea caused by the impact using the multiple measurements of the displacement of the cornea; and
calculating an internal pressure value of the eye using the calculated damping factor.

17. A method according to claim 14, wherein performing multiple measurements of the displacement is done using at least one optical sensor is, the optical sensor is selected from at least one of: a confocal chromatic sensor, a laser doppler vibrometer or a laser displacement sensor.

18. A method according to claim 14, wherein performing multiple measurements of the displacement is done using at least one optical sensor comprising at least one light emitting diode (LED) and at least one photosensor, wherein the at least one photosensor is selected from at least one of: a phototransistor, or a photodiode.

19. A method according to claim 14, further comprising
measuring a distance between the cornea of the eye and a tonometer and
using the measured distance as a feedback signal for moving the tonometer at a desired position relative to the cornea.

20. A method according to claim 14, wherein the impact means is one of: a solid probe or at least one drop of liquid.
